(19) United States
(12) Patent Application Publication
(10) Pub. No.: US 2015/0264952 A1
(43) Pub. Date: Sep. 24, 2015

(54) CARBOHYDRATE COMPOSITION OBTAINABLE FROM MEDITERRANEAN FRUITS

(71) Applicant: WILD Valencia S.A., Valencia (ES)

(72) Inventors: Hans-Peter Wild, Eppelheim (DE); Rafael Salom, L'Alcudia (ES); Ignacio Zaldua, Valencia (ES)

(21) Appl. No.: 14/729,227

(22) Filed: Jun. 3, 2015

Related U.S. Application Data
(62) Division of application No. 12/138,235, filed on Jun. 12, 2008.

(30) Foreign Application Priority Data
Jun. 13, 2007 (EP) 07011615.7

(51) Int. Cl. A23C 9/13; A23L 2/60; C13K 13/00; A61K 31/70; C13K 1/00; C13K 11/00; A23L 2/38; A23L 2/02

(52) U.S. Cl. CPC A23C 9/13; A23L 2/38; A23L 2/60; A23L 2/02; A61K 31/70; C13K 1/00; C13K 11/00; C13K 13/00; A23V 2002/00

(57) ABSTRACT

The present invention is directed to a composition comprising carbohydrates obtainable from carob and carbohydrates obtainable from at least one further fruit.

The present invention is directed to a composition comprising carbohydrates obtainable from carob and carbohydrates obtainable from at least one further fruit.

The use of natural fruit-derived products has become more important in recent years in view of increasing health consciousness.

In order to contribute to the human health it is desirable that foodstuff has a low glycaemic index (GI) within a balanced diet and a healthy lifestyle. The GI was first introduced in 1981 and is a classification of the blood glucose raising potential of carbohydrate foods. It is defined as the incremental area under the blood glucose curve of a 50 g carbohydrate portion of a test food expressed as a percentage of the response to 50 g of a reference food taken by the same subject on a different day.

Carbohydrate foods consumed in isoglucidic amounts produce different glycaemic responses depending on many factors, such as particle size, cooking and food processing, other food components (e.g., fat, protein, dietary fibre) and starch structure. The principle is that the slower the rate of carbohydrate absorption, the lower the rise of blood glucose level and the lower the GI value. High-GI foods are characterised by fast-release carbohydrate and higher blood glucose levels. A GI value ≧70 is considered high, a GI value 56-69 inclusive is medium and a GI value ≦55 is low (where glucose=100).

The GI of foods has important implications for the prevention and treatment of the major causes of morbidity and mortality in Western countries, including Type 2 Diabetes, cardiovascular disease, coronary heart disease, obesity, and hyperlipidemia. In addition, low GI foods have been associated with prolonged endurance during physical activity, improved insulin sensitivity and increase in colonic fermentation.

The problem underlying the present invention is to provide a composition allowing the preparation of foodstuff having a low GI and a pleasant taste.

Said problem is solved by a composition, comprising carbohydrates obtainable from carob and carbohydrates obtainable from at least one further fruit.

In a preferred embodiment the further fruit is a Mediterranean fruit.

In another preferred embodiment the composition comprises carbohydrates obtainable from carob and apple, more preferably 40-60% (w/w) carbohydrates obtainable from carob, and 40-60% (w/w) carbohydrates obtainable from apple, wherein the percentages are based on the dry matter.

The composition comprises in a further preferred embodiment carbohydrates obtainable from carob, apple and grape, more preferably 35-45% (w/w) carbohydrates obtainable from carob, 40-50% (w/w) carbohydrates obtainable from apple, and 10-20% (w/w) carbohydrates obtainable from grape, in particular 40% (w/w) carbohydrates obtainable from carob, 45% (w/w) carbohydrates obtainable from apple, and 15% (w/w) carbohydrates obtainable from grape, wherein the percentages are based on the dry matter. The grape is preferably white grape.

Preferably, the composition comprises carbohydrates obtainable from carob, apple, grape and pear, in particular 35-45% (w/w) carbohydrates obtainable from carob, 35-45% (w/w) carbohydrates obtainable from apple, 5-15% (w/w) carbohydrates obtainable from grape, and 5-15% (w/w) carbohydrates obtainable from pear, wherein the percentages are based on the dry matter.

In another preferred embodiment the composition comprises carbohydrates obtainable from carob, apple, grape, pear, peach, orange and lemon, in particular 30-40% (w/w) carbohydrates obtainable from carob, 35-45% (w/w) carbohydrates obtainable from apple, 5-15% (w/w) carbohydrates obtainable from grape, 5-15% (w/w) carbohydrates obtainable from pear, 1-3% (w/w) carbohydrates obtainable from peach, 1-3% (w/w) carbohydrates obtainable from orange, and 0.5-1.5% (w/w) carbohydrates obtainable from lemon, wherein the percentages are based on the dry matter.

In a particular preferred embodiment the composition comprises 35% (w/w) carbohydrates obtainable from carob, 40% (w/w) carbohydrates obtainable from apple, 10% (w/w) carbohydrates obtainable from white grape, 10% (w/w) carbohydrates obtainable from pear, 2% (w/w) carbohydrates obtainable from peach, 2% (w/w) carbohydrates obtainable from orange, and 1% (w/w) carbohydrates obtainable from lemon, based on the dry matter.

The term carbohydrate as used herein designates chemical compounds containing oxygen, hydrogen, and carbon atoms, in particular sugars such as mono- and di-saccharides, and polyalcohols.

The composition comprises preferably glucose, fructose, saccharose, polyalcohols, and other sugars, in particular 18-28% (w/w) of glucose, 30-44% (w/w) of fructose, 16-33% (w/w) of saccharose, 7-13% (w/w) of polyalcohols, and 1-3% (w/w) of other sugars, wherein the percentages are based on the dry matter. These ranges depend on the specific fruits used for preparing the composition (e.g., variety, crop features) and are subject to biological variations.

In a preferred embodiment the composition of the invention is in the form of a syrup. If the composition is present in the form of a syrup the water content is preferably 21-31% (w/w).

The composition of the invention is prepared by a process comprising
a. obtaining a composition comprising carbohydrates from carob,
b. obtaining a composition comprising carbohydrates from at least one further fruit, and
c. and mixing the products of steps a) and b).

The composition obtained in step a) comprises preferably carbohydrates form carob pulp. The carob pulp used in the process is preferably cut, seedless and sieved to a homogeneous size. The size of the seedless carob pulp is preferably 10-20 mm. The moisture content of the seedless carob pulp is preferably 5 to 15%.

The carob can be extracted as described in U.S. Pat. No. 5,624,500. The content of soluble solids expressed in ° Brix ranges preferably from about 16-22° Brix in the obtained raw extract. The pH of the raw carob extract is preferably between 4.6-5.8.

Preferably the aqueous carob extract is decanted and centrifuged, and the insoluble parts are removed in step a) of the process. The carob extract is preferably first decanted and subsequently centrifuged. The centrifuge is preferably a vertical centrifuge. The content of initial insoluble solids of the raw carob extract is preferably reduced until less than 0.8% (v/v).

In a further preferred embodiment, this aqueous carob extract is pasteurized and/or clarified by means of ultrafiltration. A plate-heat exchanger is preferably used for pasteurisation. The temperature for pasteurisation is preferably 85+/−2° C. The pasteurisation time is preferably at least 20 seconds.

The clarification is preferably performed by ultrafiltration with a ceramic membrane which preferably has a porous size of about 75,000 Daltons (molecular weight cut-off). The temperature during clarification is preferably below 40° C. The clear carob extract has preferably 14-20° Brix.

In a preferred embodiment of the process the at least one fruit in step b) is selected from the group consisting of apple, white grape, pear, peach, orange, and lemon. Preferably, carbohydrates are obtained from orange pulp in step b).

The orange pulps are preferably milled, water extracted and pressed in step b) of the process. The milling of the orange pulps is preferably performed by a hammer-mill. The particle size of the orange pulp after milling is preferably 2-3 mm. The ratio of water to orange pulp for the water extraction is preferably around 2.5:1. The extraction lasts preferably about 30 minutes. The extraction is preferably a continuous extraction. For pressing a belt press is preferably used.

The obtained aqueous extract of orange has preferably between 3-5° Brix. The pulp content of the aqueous extract is preferably around 10-15% (v/v). The pH of the aqueous extract is preferably <4.0. This aqueous is preferably decanted and centrifuged, and the insoluble parts are removed. The initial pulp content of the raw orange extract is preferably until 1-2% (v/v).

In a further preferred embodiment this extract is pasteurized and/or clarified by ultrafiltration. Pasteurisation is preferably performed in a plate-heat exchanger. The pasteurisation temperature is preferably 85+/−2° C. The pasteurisation time is preferably at least 20 seconds. The clarification is preferably performed by ultrafiltration with a membrane having a porous size of about 75,000 Daltons (molecular weight cut-off). The membrane is preferably a ceramic membrane. The temperature during clarification is preferably below 40° C. The normal contents of soluble solids of the clear orange extract ranges preferably from 2-4° Brix.

The clear orange extract is preferably concentrated to 60-65° Brix. The concentration is preferably performed in a falling-film evaporator under vacuum conditions.

The composition in step b) of the process comprises preferably carbohydrates in the form of a fruit juice concentrate. The fruit juice concentrate is preferably a clear fruit juice concentrate and has the following analytical characteristics:

The clear apple juice concentrate has preferably 69-71° Brix.

The clear white grape juice concentrate has preferably 64-66° Brix.

The clear pear juice concentrate has preferably 69-71° Brix.

The clear peach juice concentrate has preferably 64-66° Brix.

The clear orange juice concentrate has preferably 64-66° Brix.

The clear lemon juice concentrate has preferably 47-50° Brix.

Such clear fruit juice concentrates are commercially available.

The mixture of step c) of the process has preferably a soluble solid concentration between 33-55° Brix. This mixture is preferably concentrated in a falling-film evaporator under vacuum conditions. For storage, the mixture is preferably concentrated to 69-71° Brix. The mixture is preferably stored at 0-5° C.

The mixture is preferably refined and decoloured by cation exchange chromatography, anion exchange chromatography, and/or adsorption chromatography. To this end, the mixture is preferably diluted with water to 20-25° Brix. A strongly acid cation exchange resin in the hydrogen form (activated) is preferably used for cation exchange chromatography. A weakly basic anion exchange resin in the hydroxide form (activated) is preferably used for anion exchange chromatography. A polymeric absorbent resin is preferably used for decolouring. In a preferred embodiment the mixture is subjected to cation exchange chromatography and anion exchange chromatography, more preferably to cation exchange chromatography, anion exchange chromatography and adsorption chromatography. Preferably, the mixture is first subjected to cation exchange chromatography, then to anion exchange chromatography, and subsequently to adsorption chromatography. The conductivity value of the mixture after adsorption chromatography is preferably at most 120 MicroSiemens/cm at 25° C.

The refined and decoloured mixture is preferably concentrated. The concentration is preferably performed in a falling-film evaporator under vacuum conditions. The mixture is preferably concentrated to 69-79° Brix.

The concentrated mixture is preferably subjected to pasteurisation. The pasteurisation is preferably performed in a plate-heat exchanger. The pasteurisation temperature is preferably 90.5-92.5° C. The pasteurisation time is preferably 45 seconds.

After pasteurisation, the concentrated mixture is preferably cooled to 10-25° C. In a preferred embodiment, the mixture is then filtered, in particular with filter having a mesh size of about 60 microns.

For storage, the concentrate is preferably cooled to 0-5° C.

The composition of the invention has preferably one or more, most preferably all of the following characteristics:

The ° Brix at 20° C. ranges preferably from 69 to 79° Brix.

The pH at 20° C. (dilution at 10-16° Brix) is preferably 3.5-4.5.

The total acidity (expressed in g/kg of concentrate) is preferably at most 1.5 g/kg (in tartaric acid).

The colour (E420 nm, dilution at 10° Brix, d=1 cm) is preferably at most 0.020.

The conductivity (MicroSiemens/cm at 25° C., in a dilution at 25° Brix) is preferably at most 150.

The Formol Index (expressed as ml NaOH 0.1N/100 ml dilution at 14° Brix) is preferably at most 1.5.

The turbidity (NTU in a dilution at 10° Brix) is preferably at most 1.5.

The composition of the invention is preferably sensorically odourless, colourless and just sweet (dilution at 10-16° Brix).

The composition of the invention is GMO-free, allergen-free, pesticide-free and heavy metals-free.

The term “osmotised water” as used herein designates water with a maximum conductivity of 10 MicroSiemens/cm at 25° C.

For the purposes of this invention, the term “extracting” is meant to comprise any separation method from a solid or liquid mixture whether it is conducted with or without a solvent.

For the purposes of this invention, the ultrafiltration, the ion exchange and the absorption (decolouring) chromatography in the process for the preparation of the composition can be performed as described in U.S. Pat. No. 6,709,527.

The so-called refining is an important step in the preparation of the composition of the invention. The refining process enables the removal of essentially all the characteristic substances from the mentioned fruit sources except the sugar and the polyalcohol species (target).

The process for preparing the composition does not involve an enzymatic or chemical step for converting carbohydrates such as oligosaccharides or polysaccharides (e.g., starch, hemicellulose, pectins) to mono- or di-saccharides or the corresponding carbohydrate monomeric units.

The composition can be used for the preparation of foodstuff, in particular a beverage, confectionery, bakery product, dairy product, ice cream or chocolate.

The composition of the invention is preferably used for the manufacture of a soft-drink, a juice beverage, a nectar, a dairy product (e.g., a whey product or a yogurt, such as a fruit yogurt or a drink yogurt), a bakery product (e.g., a cookie, a muffin, a cereal bar, a fruit bar, or a cereal breakfast coating), a confectionery (e.g., a jelly, a soft candy, a pectin-jelly candy, or a hard-boiled candy), an ice cream, or chocolate (e.g., a chocolate bar or a chocolate sauce).

More specifically, the composition of the present invention can be used for the following foodstuff:
Soft-Drinks
Beverages (sparkling or still).
Drinks, such as juice-containing drinks.
Nectars, by replacing the used regular sugars.
Clear or cloudy beverages for all the aforementioned categories.
Tea Drinks
By replacing the used regular sugars.
Functional Drinks
By replacing the used regular sugars and also being combined with artificial sweeteners (e.g., saccharin, acetosulfame, aspartame, cyclamate, or sucralose).
Sport Drinks
By replacing the used regular sugars.
Herbal Drinks
By replacing the used regular sugars and also being combined with artificial sweeteners (e.g., saccharin, acetosulfame, aspartame, cyclamate, or sucralose).
CONFECTIONERY: by replacing the used regular sugars.
Jelly Gums.
Soft Candies.
Pectin Candies/Pectin Jellies.
Chewing Gums.
Hard Boiled Candies.
CHOCOLATES: by replacing the used regular sugars.
Dark Chocolate bars.
Chocolate sauces.
Chocolate Pralinés: the chocolate base and the fillings or also the fruit fillings.
Fruit Preparations, e.g., for fillings, toppings etc. of the chocolate products; by replacing the used regular sugars.
BAKERY: by replacing the used regular sugars.
Soy Bars.
Cereal Bars.
Fruit Bars: optionally with fillings such as fruit fillings
Muffins.
Breakfast Cereal Coatings.
Biscuits: optionally with fillings such as fruit fillings.
Cookies: optionally with toppings such as fruit toppings.
Fruit Preparations, e.g. for fillings, toppings, etc. of the bakery products; by replacing the used regular sugars.
ICE CREAMS, WATER ICE & SORBETS: by replacing the used regular sugars. No fat, low fat and whole fat categories.
DAIRY: by replacing the used regular sugars. No fat, low fat and whole fat categories.
White Yogurts.
Fruit Yogurts.
Drink Yogurts.
Fruit Drink Yogurts.
Whey Drink.
Fruit Whey Drink.
All the Fruit Preparations to be used in the aforementioned dairy products: by replacing the used regular sugars.
JAMS/MARMALADES: by replacing the used regular sugars.

The composition of the invention represents a semi-finished product and allows for the preparation of low GI foodstuff. The composition of the invention significantly helps to reduce the GI of the foodstuff. However, since the GI of the final product may be altered by further ingredients, the final product will not necessarily have a low GI.

In a further embodiment the invention is directed to a pharmaceutical composition comprising the composition of the invention. The composition of the invention is preferably used for the prevention or treatment of type 2 diabetes, cardiovascular disease, coronary heart disease, obesity or hyperlipidemia.

The composition of the invention has been subjected to GI—clinical tests in several specialised centres (e.g., Oxford Brookes University, Profil Institute Neuss, Freiburg University) in order to determine its GI and the GI of some foodstuff applications. These tests indicated a GI ranging from 34 to 42, depending on the used fruit percentage of the formula. The tests were performed following an official method specified by the FAO/WHO (“Carbohydrates in Human Nutrition”. FAO Food and Nutrition Paper 66. Report of a Joint FAO/WHO Expert Consultation. FAO, Rome 1998).

The clinical tests show that the composition of the invention is suitable for treating or preventing type 2 diabetes, cardiovascular disease, coronary heart disease, obesity or hyperlipidemia.

In beverage/drink applications the GI as determined by the clinical tests ranged from 34 to 54, depending on the recipe. These results were obtained for applications wherein the composition of the invention was used together with other food ingredients, even if other carbohydrate sources (e.g. fruit juice concentrates) were used.

In all cases the obtained GIs were classified as low. Thus, the composition of the invention significantly lowers the GI of foodstuff when it is applied in foodstuff, in comparison with the same foodstuff formulated and prepared by using regular sugar sources (e.g., glucose syrup, crystal sugar-sucrose).

The composition allows for the preparation of foodstuff with surprisingly low GI.

A beverage/drink application having a GI of 34 can be prepared for example from a composition comprising 40% (w/w) carbohydrates obtainable from carob, 45% (w/w) carbohydrates obtainable from apple, and 15% (w/w) carbohydrates obtainable from grape, wherein the percentages are based on the dry matter.

A beverage/drink application having a GI of 42 can be prepared for example from a composition comprising 35% (w/w) carbohydrates obtainable from carob, 40% (w/w) carbohydrates obtainable from apple, 10% (w/w) carbohydrates obtainable from grape, 10% (w/w) carbohydrates obtainable from pear, 2% (w/w) carbohydrates obtainable from peach, 2% (w/w) carbohydrates obtainable from orange, and 1% (w/w) carbohydrates obtainable from lemon, wherein the percentages are based on the dry matter.

The composition can replace completely or partially the used regular sugars in food applications (e.g., crystal sugar or saccharose, fructose syrup, glucose-fructose syrups, glucose syrups).

Drinks/beverages prepared from the composition of the invention are preferably diluted for human consumption until the proper dilution factor based on the desired sweetening strength and/or sensorial targets (e.g., with water, fruit juices, or other liquid ingredients).

In case of other food systems (e.g., confectionery, bakery, dairy, ice cream, chocolate), the composition of the invention can be applied directly (in concentrate form) or blended with other characteristic ingredients of the specific food application during the process.

In all the cases, the dosage depends on the required sweetening strength/profile or the targeted final taste.

The composition of the invention has a well-balanced sweet taste and a particular mouth-feel. Further, the taste is weakly fruity and full-bodied compared with regular sugars used in the food industry (e.g., crystal sugar, glucose syrup, invert sugar syrup, fructose syrup).

The composition of the invention is free of odour and colour.

The production process of the fruit extracts and the composition of the invention will be described in the following with reference to preferred embodiments.
A. Description of preferred production process of fruit extracts of carob and orange
B. Description of preferred production process of the composition of the invention from mixed fruit extracts

In the following the preferred steps of preparing the claimed composition are described in more detail.

### A.—Fruit Extracts

All the material and technological aids used are strictly “food grade” approved.

### A.1. Carob Extract

### A.1.1 Incoming Raw Material: Seedless Carob Pulp

Seedless carob pulp is preferably used as raw material for the carob extract production. The seedless carob pulp has preferably an average particle size of 10-20 mm and a moisture content of 5-15%.

The average composition of the seedless carob pulp is as follows:
Fats (%) 0.14
Proteins (%) 3-4
Soluble Carbohydrates (%) 40-50
Hemicelluloses (%) 17-19
Lignine (%) 18-20
Celluloses (%) 8-10
Ash (%) 2-3
Tannins (Polyphenols) (%) 1-3
Magnesium (ppm) 225-250
Calcium (ppm) 2800-3000
Potassium (ppm)

The percentages are expressed in weight of the dry matter.

### A.1.2 Extraction

For the purposes of this invention, the carob can be extracted as described in U.S. Pat. No. 5,624,500.

The content of soluble solids expressed as ° Brix ranges preferably from about 16-22° Brix in the obtained raw carob extract.

The regular pH of the raw carob extract is preferably between 4.6-5.8.

### A.1.3 Decantation—Centrifugation

By using decanters (first) and vertical centrifuges (second), the content of initial insoluble solids of the raw carob extract is preferably reduced until less than 0.8% (v/v).

### A.1.4 Pasteurisation

Micro-stability and inactivation of endogenous enzymes are the main targets of this step.

It is preferably achieved in a regular plate-heat exchanger pasteurisation unit with the following thermal parameters:
Temperature: 85+/−2° C.
Time: minimum 20 seconds.

Once the raw carob extract has been pasteurised, it is preferably rapidly chilled until 15-20° C.

### A.1.5 Clarification

The Clarification process of the raw carob extract is achieved by means of ultrafiltration (U-F).

The used U-F equipment has preferably a membrane porous size of about 75,000 Daltons (molecular weight cut-off), ceramic membranes, vertical configuration.

The used membranes are preferably “food grade approved” by conforming to 3-A Sanitary Standards for cross-flow membranes modules: 45-02 and under authorisation number 1181.

The maximum working temperature of the filtration system is preferably below 40° C. on the product.

The U-F permits obtaining a clear-brilliant-transparent clear carob extract, with dark brownish colour and typical carob odour. The normal content of soluble solids of this clear liquid fraction ranges preferably from 14-20° Brix.

The permeate—clear carob extract (14-20° Brix) is preferably stored in tanks for being mixed with clear fruit juice concentrate and/or a clear orange extract concentrate (produced according to chapter A.2).

### A.2. Orange Extract

### A.2.1 Incoming Raw Material: Orange Pulp

Orange pulp is the used raw material for the orange extract production. The incoming orange pulp contains different non-edible parts of the orange fruit once the juice has been obtained by squeezing the orange: albedo, flavedo, juice sacs are the main constituents of this raw material.

The orange pulp is preferably initially checked in terms of moisture/humidity (average: 75-85% humidity) and sound aspect (e.g., free of non-orange material, no fermented pulp, typical fresh orange pulp odour).

### A.2.2 Milling—Grinding

The orange pulp is preferably transported through screw-conveyors for feeding two hammer-mill equipments which are crushing, grinding and sieving the orange pulp until a regular particle size of 2-3 mm is achieved.

### A.2.3 Extraction—Pressing

The milled-ground orange pulp (of the step A.2.2) is preferably fed into a tank and mixed with osmotised water at the ratio of “water:orange” of around 2.5:1. The tank contains preferably a stirrer which facilitates the homogeneous extraction of the orange pulp's soluble components. After about 30 minutes of continuous extraction and stirring, a homogenous and thick orange paste is obtained.

The extraction is preferably completed by feeding the orange paste to a belt-press which enables to separate a liquid fraction (named raw orange extract) and a solid fraction (named orange press-cake). Orange press-cake is considered as a waste material.

The raw orange extract is preferably a very pulpy/cloudy orange coloured liquid with between 3-5° Brix. The pulp content is preferably around 10-15% (v/v). The pH is preferably <4.0.

### A.2.4 Decantation—Centrifugation

By using proper decanters (first) and vertical centrifuges (second), the initial pulp content of the raw orange extract is preferably reduced until 1-2% (v/v).

### A.2.5 Pasteurisation

Pasteurisation is preferably achieved in a regular plate-heat exchanger pasteurisation unit with the following thermal parameters:
Temperature: 85+/−2° C.
Time: minimum 20 seconds.

The main target of this step is to increase the microstability.

Once the orange extract has been pasteurised, it is preferably rapidly chilled until 15-20° C.

### A2.6 Clarification

The clarification process of the “pulp reduced” orange extract is achieved by means of ultrafiltration (U-F).

For the purpose of this invention, this clarification step by using ultrafiltration can be performed as described in chapter A.1.5.

The U-F permits obtaining a clear-brilliant-transparent clear orange extract, with yellowish colour and typical orange odour. The normal contents of soluble solids of this clear liquid fraction ranges preferably from 2-4° Brix.

The permeate, i.e., the clear orange extract, is preferably stored in tanks to be rapidly processed.

### A.2.7 Concentration and Storage

The clear orange extract is preferably concentrated in a falling-film evaporator under vacuum conditions.

The clear orange extract is preferably concentrated from 2-4° Brix until between 60-65° Brix.

Finally, the obtained clear orange extract concentrate is preferably stored in tanks in cool conditions (0-5° C.) for a further processing step.

A.3. Blending/Mixing the Clear Carob Extract with Clear Orange Extract Concentrate and/or Fruit Juice Concentrates and Concentration: Clear Fruit Concentrate Blend.

### A.3.1 Blending/Mixing

The clear carob extract with preferably between 14-20° Brix (see chapter A.1) is mixed with:
clear orange extract concentrate with preferably between 60-65° Brix (see chapter A.2), and/or
with at least one clear fruit juice concentrate.

The clear fruit juice concentrates have preferably the following analytical characteristics:
Clear Fruit Juice Concentrate ° Brix at 20° C.
Clear Apple Juice Concentrate 69-71
Clear White Grape Juice Concentrate 64-66
Clear Pear Juice Concentrate 69-71
Clear Peach Juice Concentrate 64-66
Clear Orange Juice Concentrate 64-66
Clear Lemon Juice Concentrate 47-50

These clear fruit juice concentrates are commercially available.

After blending or mixing, the product reaches preferably a soluble solid concentration between 33-55° Brix.

### A.3.2 Concentration and Cool Storage

The product blend/mix obtained in the foregoing step and belonging at the chapter A.3.1 is now preferably concentrated in a falling-film evaporator under vacuum conditions.

The product blend/mix is preferably concentrated from 33-55° Brix until between 69-71° Brix.

Finally, the obtained product blend/mix is preferably stored in tanks in cool conditions (0-5° C.) for a further processing step.

### B.—The Composition

B.1 Dilution with Osmotised Water.

The product blend/mix produced in the foregoing chapter A.3.2 is preferably diluted with osmotised water until between 20-25° Brix.

### B.2 Refining and Decolouring: Ion-Exchange and Adsorption Technology (Respectively).

Refining and decolouring is preferably performed as follows:
First: Cation exchange treatment by using activated cationic resins.
Second: Anion exchange treatment by using activated anion resins.
Third: Adsorption/decolouring treatment by using activated polymeric resins.

Preferably, a continuous conductivity monitoring “in line” is providing conductivity data (expressed in MicroSiemens/cm at 25° C.).

The conductivity value is preferably at most 120 MicroSiemens/cm at 25° C., measured in the outlet of the adsorption/decolouring resin column (third step of the treatment).

The de-ionised and decoloured product is preferably stored in tanks.

### B.3 Concentration and Storage

The de-ionised and decoloured product obtained in the foregoing step (see chapter B.2) is preferably concentrated in a falling-film evaporator under vacuum conditions.

The de-ionised and decoloured product is preferably concentrated from 15-20° Brix until between 69-79° Brix.

Finally, the concentrated product is preferably stored in tanks in cool conditions (0-5° C.) for the further final processing steps, preferably pasteurization, filtration and final storage.

### B.4 Pasteurisation, Filtration, Final Storage

Before the filling-up or packaging, the composition is preferably microbiologically stabilised by:
Pasteurisation: 90.5-92.5° C. during minimum 45 seconds.
Cooling/Chilling: 10-25° C.
Filtration: 60 microns (mesh screen size).

The pasteurisation equipment consists preferably in a plate-heat exchanger and a holding system.

The cooling process is preferably performed with a plate-heat exchanger.

The composition of the invention is preferably stored in cool conditions (cold warehouse) at 0-5° C.

The composition of the invention is sensorically odourless and nearly colourless (from colourless until a very weak pale yellow colour in concentrated form, but colourless in diluted form, e.g., 10-16° Brix).

The composition of the invention has preferably one or more, most preferably all of the following physico-chemical characteristics:
The ° Brix at 20° C. ranges preferably from 69 to 79° Brix.
The pH at 20° C. (dilution at 10-16° Brix) is preferably 3.5-4.5.
The total acidity (expressed in g/kg of concentrate) is preferably at most 1.5 g/kg (in tartaric acid).
The colour (E420 nm, dilution at 10° Brix, d=1 cm) is preferably at most 0.020.
The conductivity (MicroSiemens/cm at 25° C., in a dilution at 25° Brix) is preferably at most 150.
The Formol Index (ml NaOH 0.1N/100 ml dilution at 14° Brix) is preferably at most 1.5.
The turbidity (NTU in a dilution at 10° Brix) is preferably at most 1.5.

Due to the process, the raw material origin and traceability, this product category is GMO-free and allergen-free. Also, due to the process (ion-exchange and adsorption technology), this product is pesticide-free and heavy metals-free.

The composition of the invention fulfills the microbiological and pathogen-free standards of the Fruit Juice Concentrates and Sugar Syrups industries.

### EXAMPLES

The weight percentages in the following examples are based on the dry matter. The final products of the following examples show a low GI.

### Example 1

### Composition of the Invention

40% (w/w) carbohydrates obtainable from carob
45% (w/w) carbohydrates obtainable from apple
15% (w/w) carbohydrates obtainable from grape

### Example 2

### Composition of the Invention

35% (w/w) carbohydrates obtainable from carob
40% (w/w) carbohydrates obtainable from apple
10% (w/w) carbohydrates obtainable from grape
10% (w/w) carbohydrates obtainable from pear
2% (w/w) carbohydrates obtainable from peach
2% (w/w) carbohydrates obtainable from orange
1% (w/w) carbohydrates obtainable from lemon

### Example 3

### **Soft Drinks Just Sweetened with Composition of the Invention

1 part of composition + 5 parts of water . . . regular sweetening strength
1 part of composition + 3.5 parts of water . . . high sweet taste.

### Example 4

### **Orange Nectar (50% Juice Content) Sweetened with Composition of the Invention Instead of Crystal Sugar (Sucrose or Saccharose)

At industrial scale: to obtain 1000 l
   90 kg of orange juice concentrate having 65° Brix.
   83.5 kg of composition having 70° Brix.
   871.5 kg of water.
Made at home:
   500 ml or 522.5 g of orange juice having 11.2° Brix.
   80 g of composition having 70° Brix.
   422.5 g of water or enough water until 1 l final volume.

### Example 5

### **White Yogurt Sweetened with Composition of the Invention

Whole fat (3.5%) White Yogurt:
   9-10 g of composition having 78° Brix
   90-91 g of white yogurt base
Low fat (1.5%) White Yogurt:
   15-16 g of composition having 78° Brix
   84-85 g of white yogurt base

1. A composition comprising carbohydrates obtainable from carob and carbohydrates obtainable from at least one further fruit.

2. The composition of claim 1, wherein the further fruit is a Mediterranean fruit.

3. The composition of claim 1 or 2, comprising carbohydrates obtainable from carob and apple.

4. The composition of claim 3, comprising 40-60% (w/w) carbohydrates obtainable from carob, and 40-60% (w/w) carbohydrates obtainable from apple, wherein the percentages are based on the dry matter.

5. The composition of any of claims 1 to 4, comprising carbohydrates obtainable from carob, apple and grape.

6. The composition of claim 5, comprising 35-45% (w/w) carbohydrates obtainable from carob, 40-50% (w/w) carbohydrates obtainable from apple, and 10-20% (w/w) carbohydrates obtainable from grape, wherein the percentages are based on the dry matter.

7. The composition of any of claims 1 to 6, comprising carbohydrates obtainable from carob, apple, grape and pear.

8. The composition of claim 7, comprising 35-45% (w/w) carbohydrates obtainable from carob, 35-45% (w/w) carbohydrates obtainable from apple, 5-15% (w/w) carbohydrates obtainable from grape, and 5-15% (w/w) carbohydrates obtainable from pear, wherein the percentages are based on the dry matter.

9. The composition of any of claims 1 to 8, comprising carbohydrates obtainable from carob, apple, grape, pear, peach, orange and lemon.

10. The composition of claim 9, comprising 30-40% (w/w) carbohydrates obtainable from carob, 35-45% (w/w) carbohydrates obtainable from apple, 5-15% (w/w) carbohydrates obtainable from grape, 5-15% (w/w) carbohydrates obtainable from pear, 1-3% (w/w) carbohydrates obtainable from peach, 1-3% (w/w) carbohydrates obtainable from orange, and 0.5-1.5% (w/w) carbohydrates obtainable from lemon, wherein the percentages are based on the dry matter.

11. The composition of any of claims 1 to 10, comprising glucose, fructose, saccharose, polyalcohols, and other sugars.

12. The composition of claim 11, comprising 18-28% (w/w) of glucose, 30-44% (w/w) of fructose, 16-33% (w/w) of saccharose, 7-13% (w/w) of polyalcohols, and 1-3% (w/w) of other sugars, wherein the percentages are based on the dry matter.

13. A process for the preparation of the composition according to any of claims 1 to 12, comprising
a. obtaining a composition comprising carbohydrates from carob,
b. obtaining a composition comprising carbohydrates from at least one further fruit, and
c. and mixing the products of steps a) and b).

14. The process of claim 13, wherein in step a) the composition comprises carbohydrates from carob pulp.

15. The process of claim 14, wherein in step a) the carob pulp is extracted with water.

16. The process of claim 15, wherein in step a) the aqueous extract of carob pulp is decanted and centrifuged, and the insoluble parts are removed.

17. The process of claim 15 or 16, wherein in step a) the aqueous extract of carob pulp is pasteurised.

18. The process of any of claims 15 to 17, wherein in step a) the aqueous extract of carob is clarified by means of ultrafiltration.

19. The process of any of claims 15 to 18, wherein in step a) the aqueous extract of carob has 14-20° Brix.

20. The process of any of claims 13 to 19, wherein in step b) the at least one fruit is selected from the group consisting of apple, white grape, pear, peach, orange and lemon.

21. The process of claim 20, wherein in step b) carbohydrates are obtained from orange pulp.

22. The process of claim 21, wherein in step b) the orange pulp is extracted with water.

23. The process of claim 21 or 22, wherein in step b) the orange pulp is milled, extracted with water and pressed, and the insoluble parts are removed.

24. The process of claim 22 or 23, wherein in step b) the aqueous extract of orange pulp is decanted and centrifuged, and the insoluble parts are removed.

25. The process of any of claims 22 to 24, wherein in step b) the aqueous extract of orange pulp is pasteurised.

26. The process of any of claims 22 to 25, wherein in step b) the aqueous extract of orange pulp is clarified by ultrafiltration.

27. The process of any of claims 22 to 26, wherein in step b) the aqueous extract of orange pulp is concentrated until 60-65° Brix.

28. The process of any of claims 13 to 27, wherein the composition in step b) comprises carbohydrates in the form of a fruit juice concentrate.

29. The process of any of claims 13 to 28, wherein the mixture in step c) is refined and decoloured by cation exchange chromatography, anion exchange chromatography, and/or adsorption chromatography.

30. The process of any of claims 13 to 29, wherein the mixture in step c) is concentrated until 69-79° Brix.

31. Use of the composition of any claims 1 to 12 for the preparation of foodstuff.

32. The use according to claim 31, wherein the foodstuff is a beverage, confectionery, bakery product, dairy product, ice cream or chocolate.

33. Pharmaceutical composition comprising the composition of any of claims 1 to 12.

34. Composition of any of claims 1 to 12 for the prevention or treatment of type 2 diabetes, cardiovascular disease, coronary heart disease, obesity or hyperlipidemia.
